(19) United States
(12) Patent Application Publication
(10) Pub. No.: US 2025/0367665 A1
(43) Pub. Date: Dec. 4, 2025

(54) SYSTEM AND METHOD FOR TRANSFERRING MATERIAL BY MEANS OF LASER RADIATION FROM A STARTING SUBSTRATE ONTO A TARGET SUBSTRATE

(71) Applicant: LPKF LASER & ELECTRONICS SE, Garbsen (DE)

(72) Inventors: Robin Alexander KRÜGER, Hannover (DE); Malte Schulz-Ruhtenberg, Wunstorf (DE)

(21) Appl. No.: 19/107,044

(22) PCT Filed: Jul. 25, 2023
(86) PCT No.: PCT/EP2023/070518

§ 371 Date: Feb. 27, 2025

(30) Foreign Application Priority Data
Aug. 31, 2022 (DE) 10 2022 122 066.4
Sep. 6, 2022 (DE) 10 2022 122 570.4

(51) Int. Cl. B01L 3/00

(52) U.S. Cl. CPC B01L 3/502761; B01L 3/50273; B01L 2200/0647; B01L 2300/069; B01L 2300/0861; B01L 2400/0406; B01L 2400/0454

(57) ABSTRACT

A system for transferring material from a source substrate to a target substrate includes the source substrate and the target substrate. The source substrate and/or the target substrate have/has a plurality of wells which are bounded by a bottom surface and one or more wall surfaces. The transfer of the material can be effected by a laser source configured to apply laser radiation.

### CROSS REFERENCE TO RELATED APPLICATIONS

This application is a U.S. National Phase application under 35 U.S.C. § 371 of International Application No. PCT/EP2023/070518, filed on Jul. 25, 2023, and claims benefit to German Patent Application No. DE 10 2022 122 066.4, filed on Aug. 31, 2022 and German Patent Application No. DE 10 2022 122 570.4, filed on Sep. 6, 2022. The International Application was published in German on Mar. 7, 2024 as WO 2024/046658 A1 under PCT Article 21(2).

### FIELD

The invention relates to a system and a method for transferring material, in particular biological materials, particles, and cells, from a source substrate to a target substrate by means of laser radiation.

### BACKGROUND

In the method known as laser-induced forward transfer (LIFT), the material is transferred from a source substrate to a target substrate by the laser radiation. This is a laser-based printing process. The method allows high-accuracy transfer of a wide range of materials, such as, for example, metals, plastics, and biomaterials, including living cells.

The LIFT method also allows processing of minute quantities of liquid since there are no dead volumes due to supply conduits. In addition, the method is very gentle on the materials to be transferred. Therefore, it is suitable in particular for positioning biomaterials.

The laser does not act directly through radiation forces as in optical tweezers, for example, but is only used as a means of controlled energy input and triggers the material transfer thermally. The materials can be embedded in different environments (matrix) as a carrier substrate.

In this method, many different parameters have to be considered. Various factors influence the process. First of all, it is possible to transfer different biological materials. The light source used also influences the process via the wavelength, the pulse duration, the beam profile, and the focus adjustment.

Moreover, there are known method variants where the laser beam does not enter at the back side of the source substrate, but passes through the target substrate and enters at the front side of the source substrate, and so the direction of material flow is opposite to the direction of the laser beam.

Various effects are used to trigger the transfer. The material to be transferred may be melted and detached from the layer by thermal expansion or by vaporized portions. The material absorbs the laser radiation either itself or is dependent on an auxiliary absorber medium.

The absorber layer may, for example, be composed of material that releases gas when exposed to radiation. This may be organic molecules that release nitrogen, such as, for example, photopolymers and triazene polymers, in particular aryltriazene photopolymers. Such a layer may be depleted by the laser radiation. The absorbing layer may be composed of material that evaporates when exposed to radiation and thus converts the energy of the laser beam into kinetic energy.

In order to form a liquid droplet, sufficient energy must be input into the system. If too little power is used, no transfer takes place. If too much power is used, many small droplets are formed and distributed as a spray on the receiver. Ideally, a directional microjet area is formed.

Depending on the material to be transferred, the receiver substrate must be prepared as well. Certain adhesive layers for materials, specialized for the production of microarrays, may be used for this purpose. In order to limit the resulting shear forces during the transfer of living cells, hydrogel layers are used as a damping layer to absorb the impact, to reduce mechanical damage, and to enable survival of the cells.

U.S. Pat. No. 7,875,324 B2 describes laser transfer of biological materials. A pulsed laser sends laser pulses into the target substrate and through the support. The pulses are absorbed by the interlayer, which causes a portion of the transfer material to be transferred to a receiving substrate. The target substrate, the receiving substrate, and the photon energy source can be moveable with respect to each other. Laser absorption and energy conversion by the interlayer results in the removal of a three dimensional pixel of biomaterial from the target and towards the receiving substrate. The technique allows for transfer of very small transferred volumes (<pL scale), spot sizes as small as 20-50 μm, and has the ability to deposit patterns of single cells.

U.S. Pat. No. 6,936,311 B2 relates to a method for depositing a biomaterial, for example, a living cell culture, on a receiving substrate. The laser energy has sufficient energy to cause the desorption of the transfer material and to deposit it on the receiving substrate. The laser-absorber layer may include gold, chrome, and titanium.

It is also known in the prior art to use microtiter plates having up to several thousands of wells, for high-throughput screenings. For large numbers of samples, the filling of such wells may be automated using pipetting robots, whose principle of operation is based on extrusion or inkjet printing, for example.

### SUMMARY

In an embodiment, the present disclosure provides a system for transferring material from a source substrate to a target substrate. The system includes the source substrate and the target substrate. The source substrate and/or the target substrate have/has a plurality of wells which are bounded by a bottom surface and one or more wall surfaces. The transfer of the material can be effected by a laser source configured to apply laser radiation.

### BRIEF DESCRIPTION OF THE DRAWINGS

Subject matter of the present disclosure will be described in even greater detail below based on the exemplary figures. All features described and/or illustrated herein can be used alone or combined in different combinations. The features and advantages of various embodiments will become apparent by reading the following detailed description with reference to the attached drawings, which illustrate the following:

FIG. 1 different relative orientations of a source substrate with respect to a target substrate, at least one of the substrates having a plurality of wells for the material;

FIG. 2 a cross-sectional view of a source or target substrate having differently contoured wells;

FIG. 3 a top view of a source or target substrate having a plurality of wells with different cross-sectional shapes;

FIG. 4 a cross-sectional view illustrating different positions of possible focal points of the laser radiation within the wells;

FIG. 5 the selective passivation of a selected cell within a well;

FIG. 6 the transfer of single cells between the source substrate and the target substrate;

FIG. 7 the transfer of single cells between the source substrate and the target substrate by means of an auxiliary material; and

FIG. 8 the introduction of absorber layers prior or subsequent to placing a cell into the well.

### DETAILED DESCRIPTION

Embodiments of the invention provide to substantially improve the transfer of, in particular, living cells, particles, or biomaterials. In particular, it is advantageously provided to enable the transfer to be performed in a more targeted and more material-friendly manner, and also with different orientations of the source substrate relative to the target substrate. It is also advantageously provided to be able to transfer a large volume without increasing the energy input.

In accordance with an embodiment of the invention, a gentle and at the same time reliable transfer of the material, in particular together with a carrier liquid, is made possible in a surprisingly simple manner by the source substrate and/or the target substrate having a plurality of contoured wells which are bounded by a bottom surface and a wall surface and configured to receive volumes of material in the picoliter and nanoliter range. Because the source substrate is provided with wells which promote, in particular concentrate or focus, the flow of material, not only is there produced an optimal microjet that avoids spray losses, but the energy required for the transfer is reduced at the same time. Moreover, the target substrate may also be provided with corresponding wells which are configured in such a way and particularly to have a cross sectional opening area so small that the entering material can be held by the action of capillary forces alone. Another particular advantage is the relatively gentle impingement of the material in the well of the target substrate, which is due to a delay caused by the capillary forces. Since the diameter of the wells needs to be only slightly greater than the droplet diameter, the air displacement during entry of the droplet can be greater, which can result in a deceleration and, thus, in a more gentle transfer.

Thus, in accordance with embodiments of the invention, material can be transferred from the source substrate to the target substrate in a surprisingly simple manner because one of the substrates can be oriented in an overhead position without any problems and without requiring any additional measures. In the overhead position of the source substrate, the material is held by the capillary forces until it is ejected by the thermal energy input of the laser. In the overhead position of the target substrate, the entering material is also held by the capillary forces. Because of this, the relative orientation of the source substrate with respect to the target substrate can even be changed during the transfer process, since the effects of gravity only have a minor impact.

For the same volume, the wells with a higher aspect ratio can be filled to a greater extent without increasing the risk of liquids mixing between the wells. On flat substrates without wells, mixing cannot be avoided because liquid media flow and, thus, would bleed into one another. Therefore, the wells enable better utilization of the surface area of the array. In addition, wells with a high aspect ratio improve the ratio of liquid surface area to volume, which reduces evaporation, for example. For the same volume, liquids in wells with a high aspect ratio have a longer optical path, which improves the sensitivity of optical examinations, for example. Wells with a small base area can be arranged at a smaller spacing relative to each other, making it possible to analyze more samples simultaneously with the same optical analysis unit.

In the case of samples on flat substrates, less surface area is available for interaction with, for example, antibodies attached to the substrate surface. This firstly reduces the absolute number of interactions and secondly also the time until a certain number of attachments have occurred. The three-dimensional surface of substrates with wells has greater contact with the volume of liquid, resulting in the formation of a greater number of attachments. This is advantageous, for example, because a detection threshold can be reached in less time or in cases where cells are stimulated with antigens, receptor interactions, etc.

The wells are not limited merely to an extremely small cross-sectional area, previously unknown in the prior art. The wall surfaces of the wells are particularly advantageously contoured in such a way that opposing wall sections are at least in some portions not parallel but inclined to each other between the bottom surface and the opening. In particular, the wells are at least in some portions not cylindrical but, for example, conical in shape, and a plurality of conical sections having different or opposite opening angles may adjoin one another in the direction of the main axis. At least one cross-sectional plane, in particular in the region of the bottom surface or of the opening of the well, may, of course, have any shape different from a circular shape, for example, an oval or polygonal shape and geometry. In such case, pairs of opposing wall sections may extend parallel or inclined to each other along the same portion of the longitudinal axis.

The wells of the source substrate and/or of the target substrate have wells which preferably have an aspect ratio (depth to width) greater than 1 or greater than 2, 3, 4, 5, 6, 7, 8, 9, 10 or higher.

The wells may preferably have a circular cross section or a rectangular cross section. The diameter of circular wells is preferably between 10 and 1000 μm or more, preferably 50 μm, 100 μm, 200 μm, 300 μm, 400 μm, 500 μm, 600 μm, 700 μm, 800 μm, 900 μm, or 1000 μm. The depth of the wells is preferably between 20 and 1100 μm or more, preferably 50 μm, 100 μm, 200 μm, 300 μm, 400 μm, 500 μm, 600 μm, 700 μm, 800 μm, 900 μm, 1000 μm, or 1100 μm.

The wells preferably have a center-to-center spacing equal to 105%, 110%, 120%, 130%, 140%, 150%, 160%, 170%, 180%, 190%, 200%, 300%, 400%, or 500% or more of the diameter or of the edge length. The distance between the edge of a well and the edge of an adjacent well is preferably 2 μm, 5 μm, 10 μm, 20 μm, 30 μm, 40 μm, 50 μm, 60 μm, 70 μm, 80 μm, 90 μm, 100 μm, 200 μm, 300 μm, 400 μm, 500 μm, 600 μm, 700 μm, 800 μm, 900 μm, or 1000 μm, or more. The density of the wells is preferably between 0.25 per mm² and 6,944 per mm², preferably 0.5 per mm², 1 per mm², 5 per mm², 10 per mm², 20 per mm², 30 per mm², 40 per mm², 50 per mm², 60 per mm², 70 per mm², 80 per mm², 90 per mm², 100 per mm², 200 per mm², 300 per mm², 400 per mm², 500 per mm², 600 per mm², 700 per mm², 800 per mm², 900 per mm², 1,000 per mm², 2,000 per mm², 3,000 per mm², 4,000 per mm², 5,000 per mm², 6,000 per mm².

When, for example, different wall sections of rotationally symmetric wells form an inclination angle with a longitudinal center axis of the well or the axis of symmetry, nozzle effects are obtained which, for the same energy input, significantly increase the flow velocity of the material to be transferred. For this purpose, the well has a throat with a cross-sectional area smaller than the bottom surface of the well.

The wall surface of the well is not limited to flat surfaces. Rather, in another preferred embodiment of the invention, individual wall sections may in some portions have a concave or convex, non-flat profile, which both optimizes the flow and enhances capillary effects. Thus, the configuration of the wall surface can determine the pressure profile.

In accordance with another preferred embodiment of the invention, the wall or bottom surface of the well of at least the target substrate is associated with an absorber layer at which the laser radiation is directed. The absorber layer may also take the form of a coating on the bottom surface or on an opposite surface on the back side.

Moreover, the absorber may also be liquid or flowable and, due to the capillary effects within the well, can be used even in an overhead position with the opening down, while at the same time ruling out the risk of accidental leakage of absorber liquid.

The absorber liquid may, of course, also be used in a usual position of the well, with the bottom surface down. In another variant, the density of the absorber liquid is greater than the density of material or of the carrier liquid, so that the absorber liquid migrates downward under the action of gravity so as to form the base layer within the well and, possibly, to displace and lift the material from the bottom surface. Once the absorber material settles to the bottom of the well and lifts the cells present there, the laser radiation can be focused thereat, which reduces the risk of the cells being damaged by the laser radiation.

Since the absorber layer may hinder the microscopic analysis of the content of the well, the absorber material can be introduced only shortly before the LIFT process is carried out. This is not easily possible with flat substrates according to the prior art since liquids can flow.

Preferably, at least some of the wells have a bottom surface which is transparent at least in some portions, so that the optical detection of the sample is effected through the bottom surface, at least a significant portion of the wells of the sample holder having an aspect ratio of the height of the well to the diameter or the edge length of the opening of greater than 1.

In a further particularly preferred embodiment of the invention, the source substrate and/or the target substrate may be configured as a glass substrate, the wells in the source substrate and/or the target substrate being formed by laser radiation. In this process, the focus of the laser radiation undergoes spatial beam shaping along a beam axis of the laser radiation, whereby modifications are produced in the glass substrate, so that the wells are then formed in the glass substrate by the action of an etching medium and by successive etching as a result of the anisotropic removal of material in the respective region of the modifications in the glass substrate. The substrate may be composed of silicon, the wells being made using the Bosch process (deep reactive ion etching). The substrate may also be made of metal, plastic, and other materials. The substrate may be made of several materials joined together, for example, of a silicon plate having wells in the form of through-holes and a glass substrate attached thereto to form the bottom of the wells. The wells may be formed by laser drilling and other material removal processes.

Furthermore, the substantially improved transfer of, in particular, living cells, particles, or biomaterials is also achieved by a method according to an embodiment of the invention where the laser beam is directed at a wall surface of the well. To this end, an embodiment of the invention makes use of the fact that the material will collect in the region of the bottom surface with a certain probability. Accordingly, a particularly material-friendly energy input is achieved by directing the laser beam at the wall surface at a distance from the bottom surface. Because of its contour, in particular the reductions or increases in cross-sectional area, the wall surface allows the material to be ejected even when the focal point of the energy input is located between the material and the opening of the well.

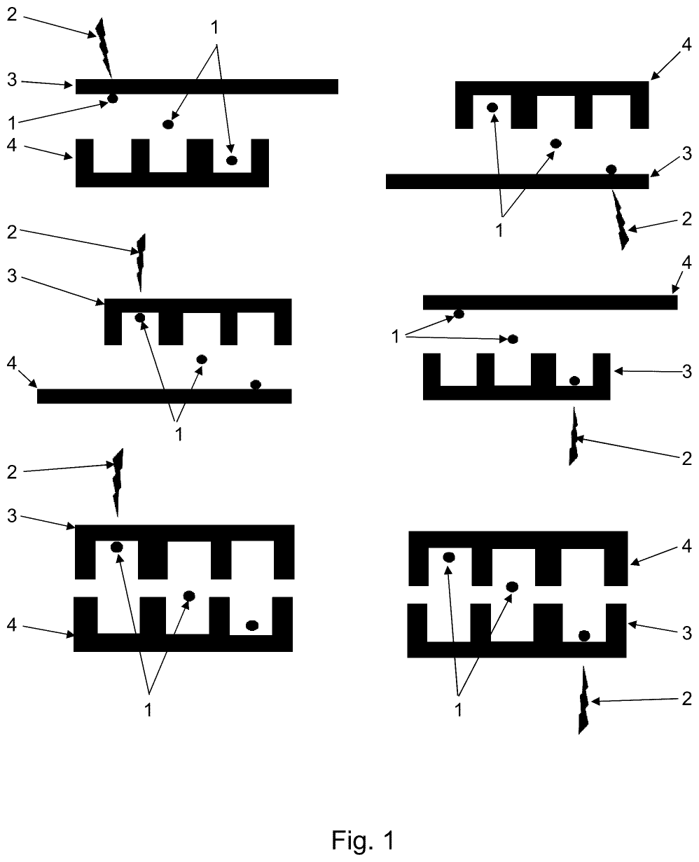

An embodiment of the invention will now be described in greater detail with reference to the figures. FIG. 1 illustrates a system for the transfer of, in particular, biological material 1, such as the single cells shown, from a source substrate 3 to a target substrate 4 by means of a device applying laser radiation 2 (a laser, e.g., a pulsed laser source), which substrates are provided with suitable wells 5 for this purpose. The inventive cross-sectional shape of well 5 firstly generates capillary forces for fixing the cells in wells 5, even in the overhead position, and secondly optimizes the ejection from well 5 of source substrate 3, for example by a nozzle effect within well 5, caused by a throat E, in order to improve the material removal with reduced thermal energy input.

This material removal is achieved using the generally known laser-induced forward transfer (LIFT) technique, where the cells are transferred, either directly or attached to beads as an auxiliary substance or transfer aid 6 as shown in FIG. 7, from well 5 or from the surface of source substrate 3 into well 5 or to the surface of target substrate 4. As shown, one of the substrates 3, 4 may also be flat. It is also possible to transfer only the beads to which are attached, for example, biomolecules of the cells, such as DNA.

It is advantageously provided for by embodiments of the invention to immobilize the cells, in particular in the direction of the large extent of substrate 3, 4. In the case of flat substrates 3, 4, the use of the LIFT technique can lead to movement of cells in the immediate vicinity of the transfer. Therefore, before each individual transfer, it must be checked where the cells are located and where laser radiation 2 is to be focused in order to prevent direct irradiation of cells or simultaneous transfer of multiple cells. The use of wells 5 makes it possible to determine the position 12 of the cells once and to thereby create a cell map. This map can then be used during the transfer to focus laser radiation 2 to suitable positions 12. Extended-duration experiments with cells always involve cell migration. Wells 5 limit this to a smaller area per cell.

Laser radiation 2 can optionally act from above or from below. In order to transfer material 1, laser energy is converted into heat, which rapidly evaporates liquid and propels material 1. The absorber 7 used for the laser energy in this process may be a medium such as water, or reagents, or added absorbers 7. The pressure propagation is channeled and concentrated by a wall surface 8 of well 5, and material 1 is focused toward opening 9 of well 5 and thereby deflected in a targeted manner.

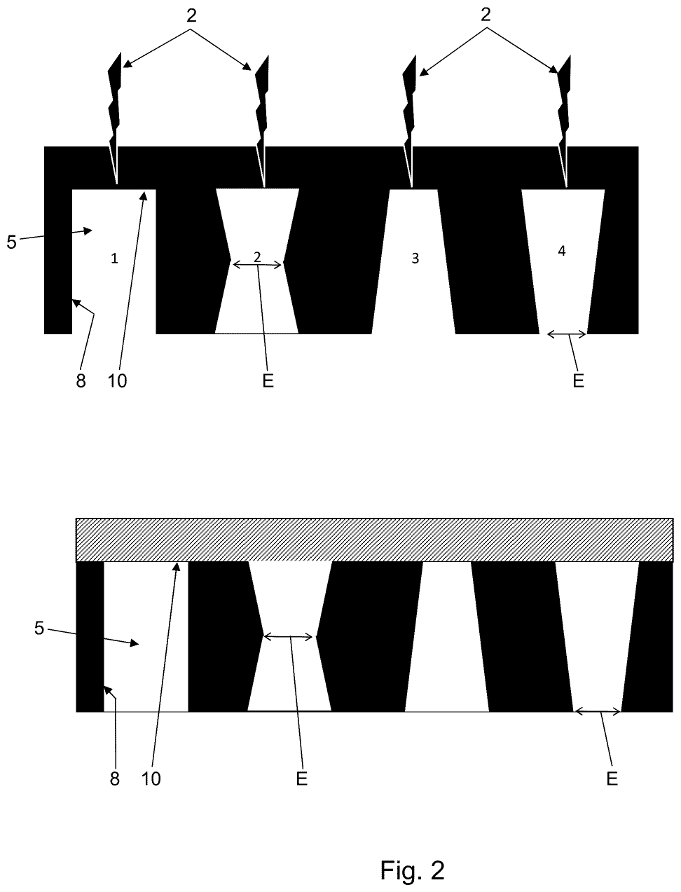

Wall surface 8 reduces the tendency to splatter, so that a larger amount of energy can be applied and thus a larger volume can be transported out of well 5 so as to increase the efficiency of the material transfer compared to a flat substrate surface. Moreover, it is virtually impossible for unwanted, unselected particles or cells to be transported because they are physically separated by wells 5. In contrast, with flat substrates according to the prior art, it is possible that transfer volumes may arise or that the material may be mixed by lateral flows. In accordance with an embodiment of the invention, the tapering cross sections with throat E, illustrated in variant 2 in FIG. 2, bring the divergent flows back together within wells 5 so that they emerge in a concentrated or focused manner. In variant 3 of the cross-sectional shapes of well 5 shown in FIG. 2, a high pressure is created in the area of a bottom surface 10, which is required to set material 1 in motion. The increasing cross-sectional area reduces the pressure so as to reduce the risk of splatter. In contrast, variant 4 shown in FIG. 2 is a cross-sectional shape that strongly focuses the material flow by the continuously decreasing cross-sectional area, and which thus provides a long path for even better focusing.

In addition to the parameters of laser power, wavelength, absorber 7, liquid for adjusting the printing process, the shape of wells 5 offers further possibilities for increasing the parameter space and optimizing the transfer of material 1 between source substrate 3 and target substrate 4, and in particular for avoiding undesired and undetected mixing of cells.

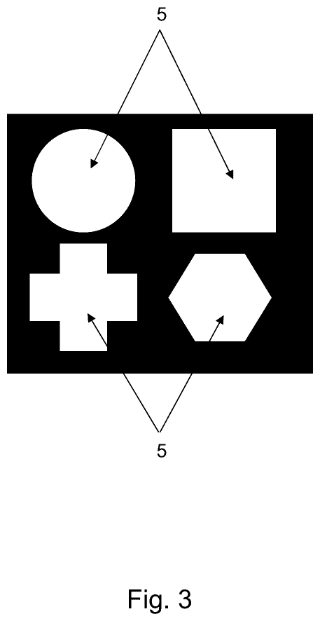

Furthermore, the different cross-sectional shapes of wells 5 illustrated in FIG. 3 provide further possibilities for influencing the transfer process with an even larger parameter space.

As can be seen in the lower portion of FIG. 2, the material properties can be adapted to the various functions, namely firstly the observation of material 1 by transparent material properties and secondly the absorption of the laser energy. Advantageously, an adaptation can also be achieved by using different lasers. The choice of laser source depends on the liquids used and on any absorber materials that may be used. Therefore, any type of laser source may be used.

For instance, pulsed laser sources may be employed. The pulse duration may be between 100 fs and 100 μs. The wavelength is selected such that laser radiation 2 is, to the extent possible, absorbed near the cells and not by the cells themselves. The absorber 7 used may, for example, be water, and a laser wavelength on the order of 2 μm may be used. Alternatively, absorber materials are used which are matched to the wavelength of the laser source. In principle, any wavelength from ultraviolet to infrared can be used.

Furthermore, glass substrate 3, 4 may, for example, be provided with an absorptive coating, for which gold is particularly suitable due to its inert and good absorption properties.

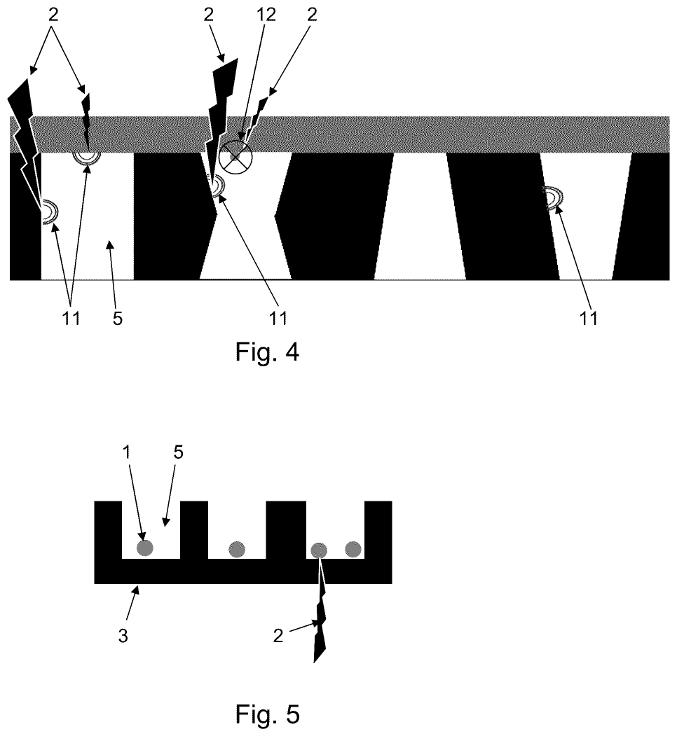

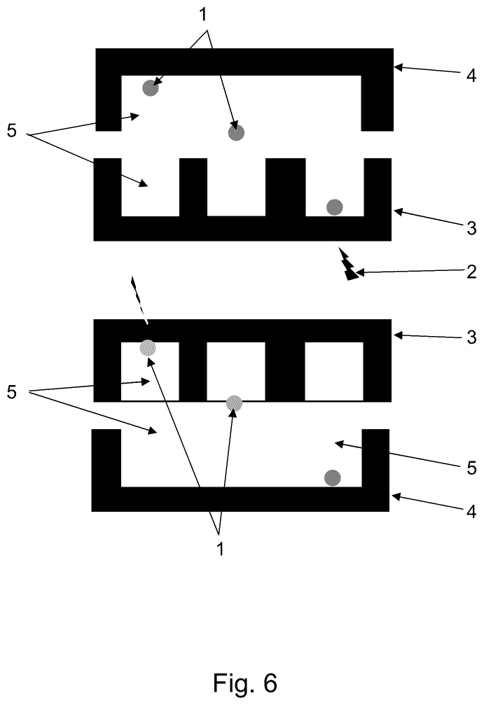

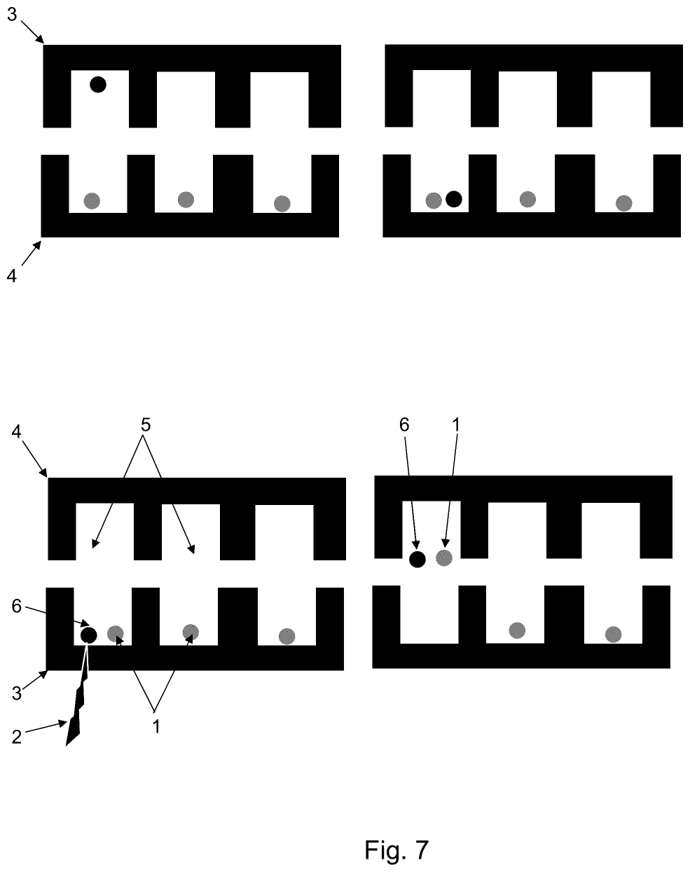

As shown in FIG. 4, the energy input can take place in different areas of bottom surface 10 or wall surface 8 of well 5. FIG. 4 illustrates the formation of a pressure bubble 11 within well 5. For some positions 12, focusing close to the cell may be undesirable because this can result in passivation of the cell, as can be seen in FIG. 5. Due to the inclination of wall surface 8, pressure bubble 11 is directed downward and reflected.

As can be seen in FIG. 7, a transfer aid 6 in the form of beads, to which the material 1 to be transported is attached, may be printed or washed into wells 5, for example by means of inkjet or LIFT techniques. This allows material 1 to be reliably transported, even with inaccurate focusing. By using a wavelength that is not absorbed by the cells, the desired transport is accomplished by means of the beads contained in wells 5, without damaging the cells.

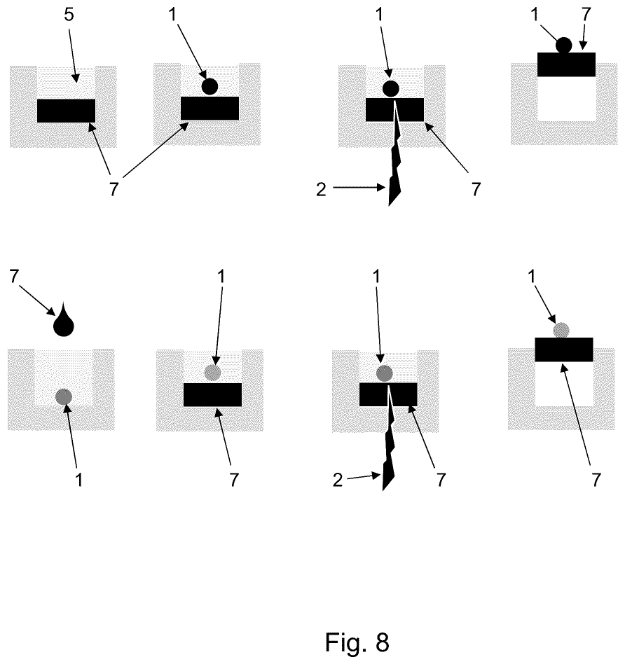

As can be seen in FIG. 8, a separate absorber layer can be created by introducing a hydrogel that contains absorber 7, which absorber layer is introduced, for example, before the cells are introduced into well 5. This is suitable in particular for fluorescent cells. The absorber layer may also be introduced after the cell. Since the absorber layer has a higher density, the cell floats up and the laser energy can be focused onto the volume of the absorber layer without the risk of damaging the cell. For this purpose, for example, a highly-concentrated sugar solution may be used, which is not introduced into well 5 until the removal takes place.

While subject matter of the present disclosure has been illustrated and described in detail in the drawings and foregoing description, such illustration and description are to be considered illustrative or exemplary and not restrictive. Any statement made herein characterizing the invention is also to be considered illustrative or exemplary and not restrictive as the invention is defined by the claims. It will be understood that changes and modifications may be made, by those of ordinary skill in the art, within the scope of the following claims, which may include any combination of features from different embodiments described above.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

### LIST OF REFERENCE CHARACTERS

1 material
2 laser radiation
3 source substrate
4 target substrate
5 well
6 transfer aid
7 absorber
8 wall surface
9 opening
10 bottom surface
11 pressure bubble
12 position
E throat

1. A system for transferring material from a source substrate to a target substrate, the system comprising:
the source substrate and the target substrate;
wherein the source substrate and/or the target substrate have/has a plurality of wells which are bounded by a bottom surface and one or more wall surfaces.

2. The system as recited in claim 1, further comprising a laser source configured to apply laser radiation to effect the transfer of the material.

3. The system as recited in claim 1, wherein the wells are configured to receive volumes of material of less than 1 microliter.

4. The system as recited in claim 1, wherein at least some of the wells in the source substrate and/or the target substrate are at least in some portions cylindrical and/or conical in shape and/or configured such that the one or more wall surfaces have/has an inclination angle of between 1° and 30° with respect to the normal to the cross-sectional plane or to the substrate plane.

5. The system as recited in claim 1, wherein at least a significant portion of the wells of the source substrate and/or the target substrate have an aspect ratio of the height of the well to the cross-sectional area of the well of greater than 1.

6. The system as recited in claim 1, wherein a size of the wells is selected such that the transferred material is held in the wells by capillary forces.

7. The system as recited in claim 1, wherein at least some wall sections have a non-flat profile with a concave and/or convex shape.

8. The system as recited in claim 1, wherein an inclination of the wall sections between the bottom surface and openings of the wells changes at least once.

9. The system as recited in claim 1, wherein each of the wells has a throat disposed between and spaced from an opening and the bottom surface and having a cross-sectional area smaller than the bottom surface of the well.

10. The system as recited in claim 1, wherein each of the wells has a non-rotationally symmetric shape along a main direction of the respective well.

11. The system as recited in claim 1, wherein the bottom surface has a coating that is absorptive to the laser radiation and/or an absorber layer.

12. The system as recited in claim 1, wherein the source substrate and/or the target substrate have/has at least in some portions a coating that is absorptive to transfer energy.

13. The system as recited in claim 1, wherein the source substrate and/or the target substrate are/is a glass substrate.

14. The system as recited in claim 1, wherein the source substrate and/or the target substrate are/is a glass substrate, and wherein the wells are formed in the glass substrate by first applying laser radiation, a focus of which undergoes spatial beam shaping along its beam axis, to produce modifications in the glass substrate along the beam axis by the laser radiation, and then forming the wells in the glass substrate by action of an etching medium and by successive etching as a result of anisotropic removal of material in respective regions of the modifications in the glass substrate.

15. A method for transferring material, the method comprising:
directing laser radiation to transfer the material from a source substrate to a target substrate, wherein the source substrate and/or the target substrate have/has a plurality of wells which are bounded by a bottom surface and one or more wall surfaces, and wherein the laser radiation is directed at the bottom surface and/or the wall surface of the at least one of the wells.

16. The method as recited in claim 15, wherein in a first step, the position of the cells is detected by a camera or an optical sensor, and the cells are then transferred, by the laser radiation being irradiated at a distance or offset from a position of each cell.

17. The system as recited in claim 1, wherein the material comprises biological materials and/or cells.

18. The system as recited in claim 6, wherein an aspect ratio of the wells is selected such that the transferred material is held in the wells by capillary forces.

19. The system as recited in claim 10, wherein each of the wells have a polygonal cross-sectional shape along the main direction of the respective well, which is orthogonal to the main plane of extent of the source substrate and/or the target substrate, and/or perpendicular to the main direction of the well.

20. The method as recited in claim 15, wherein the material comprises biological materials and/or cells.
